(19) United States
(12) Patent Application Publication
(10) Pub. No.: US 2012/0264110 A1
(43) Pub. Date: Oct. 18, 2012

(54) AUTOMATED PAP SCREENING USING A PLURALITY OF BIOMARKERS AND MULTI-SPECTRAL IMAGING

(76) Inventors: Elliot S. Wachman, Lakewood, NJ (US); Stanley J. Geyer, Pittsburgh, PA (US); Jill Wachman, Lakewood, NJ (US)

(21) Appl. No.: 13/434,084

(22) Filed: Mar. 29, 2012

Related U.S. Application Data
(60) Provisional application No. 61/474,524, filed on Apr. 12, 2011.

(51) Int. Cl. C12Q 1/70; C12Q 1/68; G01N 33/567

(52) U.S. Cl. 435/5; 435/7.21; 435/6.1

(57) ABSTRACT

An automated screening method for detecting abnormalities in a sample. The method includes steps of staining a sample with a histologic or cytologic stain for transmission light microscopy to provide a stained sample; exposing the stained sample to a plurality of differentially-labeled biomarkers, wherein each of the biomarkers is labeled with a distinct transmission stain or fluorescence probe; at a first location, generating at least one multi-spectral image of the stained sample using signals obtained from the plurality of differentially-labeled biomarkers and the histologic or cytologic stain; and automatically determining whether the first location requires further pathologist review or interpretation.

### CROSS-REFERENCE TO RELATED APPLICATIONS

This application claims priority to U.S. provisional patent application Ser. No. 61/474,524, filed Apr. 12, 2011, the entire contents of which are incorporated herein by reference.

### FIELD OF THE INVENTION

Embodiments of the invention relate to automated papanicolaou (Pap) screening for the prognosis and diagnosis of cervical atypical cells, cervical intraepithelial lesions, and cervical cancer.

### BACKGROUND

Cervical cancer is a leading cause of death of women. In the U.S., approximately 13,000 women were diagnosed with cervical cancer in the year 2002 alone. The Pap test which involves Pap staining cells and then examining the Pap stained cells with a conventional light microscope is commonly used to screen for cervical cancer, and is generally considered to be the most effective screening technique ever developed for any cancer. Current medical practice calls for each female above adolescence to receive one Pap test annually. This amounts to approximately 50 million tests a year in the U.S., and approximately 60 million abroad.

Of the Pap tests done each year in the U.S., approximately 7% display abnormalities that require additional clinical follow-up. A substantially greater percentage reveal other abnormalities that do not necessarily represent precancerous changes, such as low grade squamous intraepithelial lesions (LSIL) and atypical squamous cells (ASC), but may nonetheless assume importance in risk stratification.

Of the nearly one hundred strains of Human Papillomavirus (HPV) that have been identified to date, a small subset has been recognized as high-risk, with a strong correlation to development of precancerous changes of the cervix known as high grade squamous inter-epithelial lesions (HSIL). Indeed, infection with a high-risk HPV constitutes the major risk factor and is a requirement for development of cervical cancer.

The conventional approach to Pap screening is not able to detect infection with high-grade HPV, nor is it able to distinguish reliably between some cases of ASC and HSIL. In response to this deficiency of conventional microscopy, the National Cancer Institute sponsored a multicenter ASCUS/LSIL Triage Study (ALTS) to determine optimum strategies for early detection of women at risk of developing cervical cancer. The triage study tested three follow up procedures: immediate colposcopy, HPV testing, and conservative management with repeating of the Pap smear examination. The trial results concluded that HPV testing is an effective option in the management of women with ASC because of its sensitivity and specificity as a disease marker.

In the ALTS study, HPV was detected utilizing a hybrid capture method in which residual fluid from liquid-based Pap smear specimens is placed into a microwell plate, and the presence of selected HPV strains produces chemiluminescence. Microscopic visualization of the infected cells in the specimen is not possible Immunohistochemical staining is an alternative approach that has been used for HPV detection. This approach allows the pathologist to visualize the infected cells, however sensitivity and specificity is reduced in comparison to the hybrid capture method. In addition, the colorimetric appearance of the sample is quite different from the customary Pap smear. In neither of these techniques is it possible to look at conventional PAP-stained cells while detecting the presence of HPV in a single slide.

Because a given Pap smear is often interpreted quite differently by different pathologists, the need for a method of quantifying diagnostic criteria has long been recognized in this profession. Automated Pap screening that relies on computer-based algorithms has the potential for addressing this problem. One fully automated screening system that has received FDA approval is the TriPath system provided by TriPath Imaging, of Burlington, N.C. The TriPath system uses morphological criteria for assessing cells that are potentially atypical or preneoplastic, choosing those cells whose nuclei are deemed to be unusually large and optically dense. However, these computational algorithms are not always capable of accurate, reliable results when confronted with the frequent occurrence of uneven staining and overlapping clumps of cells.

### SUMMARY

This Summary is provided to comply with 37 C.F.R. §1.73, presenting a summary of the invention to briefly indicate the nature and substance of the invention. It is submitted with the understanding that it will not be used to interpret or limit the scope or meaning of the claims.

Embodiments of the present invention describe automatic PAP screening methods for detecting abnormalities in cervical cells. The cervical cell sample is first collected in a liquid-based fixative and then stained with PAP stain to help visualize the cell morphology. A plurality of biomarkers in the PAP-stained cells are then labeled using distinct fluorescence probes or transmission stains, at least one of the plurality of different labeled biomarkers operable for targeting at least one proliferative biomarker when present and others of the plurality of different labeled biomarkers operable for labeling respective ones of a plurality of different high-risk human papilloma virus (HPV) strains when present. Multi-spectral images of the cell samples are generated using signals obtained from the plurality of labeled biomarkers and from the Pap stain. The multi-spectral images can include images from both visible and near infrared wavelengths, determined by the specific choice of labels used. Automated analysis of the multispectral biomarker images will determine from which locations higher magnification multispectral PAP images should be acquired and analyzed for subsequent review by a pathologist.

In one embodiment, the invention provides an automated screening method for detecting abnormalities in a sample. The method includes steps of staining a sample with a histologic or cytologic stain for transmission light microscopy to provide a stained sample; exposing the stained sample to a plurality of differentially-labeled biomarkers, wherein each of the biomarkers is labeled with a distinct transmission stain or fluorescence probe; at a first location, generating at least one multi-spectral image of the stained sample using signals obtained from the plurality of differentially-labeled biomarkers and the histologic or cytologic stain; and automatically determining whether the first location requires further pathologist review or interpretation.

In another embodiment, the invention provides an automatic Pap screening method for detecting abnormalities in a cervical cell sample. The method includes steps of staining a cervical cell sample with papanicolaou (Pap) stain to provide Pap stained cells; exposing the Pap stained cells to a plurality of differentially-labeled biomarkers, at least one of the plurality of differentially-labeled biomarkers operable for labeling at least one proliferative marker and at least one other of the plurality of differentially-labeled biomarkers operable for labeling at least one of a plurality of different high-risk human papilloma virus (HPV) strains, wherein each of the biomarkers is labeled with a distinct transmission stain or fluorescence probe; at a first location, generating at least one multi-spectral image of the Pap stained cells using signals obtained from the plurality of differentially-labeled biomarkers and the Pap stain, and automatically determining whether the first location includes at least one of a high-risk HPV strain and a proliferative marker using the multi-spectral image.

### DETAILED DESCRIPTION

The present invention is described with reference to the attached figures, wherein like reference numerals are used throughout the figures to designate similar or equivalent elements. The figures are not drawn to scale and they are provided merely to illustrate the instant invention. Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. In other instances, well-known structures or operations are not shown in detail to avoid obscuring the invention. The present invention is not limited by the illustrated ordering of acts or events, as some acts may occur in different orders and/or concurrently with other acts or events. Furthermore, not all illustrated acts or events are required to implement a methodology in accordance with the present invention.

Embodiments of the invention are directed to methods for diagnosing cervical disease. Such methods are generally able to specifically identify high-grade cervical disease that is present in patient populations, including those cases classified as LSIL or CIN1 by Pap staining that are actually high-grade disease (i.e., “false negatives”).

The biomarkers used in embodiments of the invention include genes and proteins, and variants and fragments thereof. In various embodiments, the biomarker may be a nucleic acid of infectious origin such as a nucleic acid sequence from a virus. Such biomarkers include DNA comprising the entire or partial sequence of the nucleic acid sequence encoding the biomarker, or the complement of such a sequence. The biomarker nucleic acids also include RNA comprising the entire or partial sequence of any of the nucleic acid sequences of interest. A biomarker protein is a protein encoded by or corresponding to a DNA biomarker of the invention. A biomarker protein comprises the entire or partial amino acid sequence of any of the biomarker proteins or polypeptides. Potential biomarkers that may be used include Her—2-neu, Ki-67, KRAS, AMACR, and CD 117.

A “biomarker” as used herein is any nucleic acid sequence or protein whose level of expression in a tissue or cell is altered compared to that of a normal or healthy cell or tissue. The term “biomarker” is intended to identify a nucleic acid sequence, gene, or protein that identifies high-grade dysplasia or the probability of a cervical abnormality progressing to high-grade dysplasia or to cancer. High-risk strains of human papillomavirus are considered to be biomarkers for the purposes of this invention. Biomarkers of the invention are selective for the presence of underlying cytological preneoplastic or neoplastic diseases of the cervix and predict either the presence of high-grade dysplasia or the ability of a lesion to advance to a high-grade lesion or to cancer. For those biomarkers that are selectively over expressed in high-grade cervical disease, they may not be over expressed in conditions classified as low-grade squamous intraepithelial lesion, atypical metaplastic squamous cells, and other conditions that are not considered to be clinical disease. Thus, detection of the biomarkers of high-grade cervical disease of the invention permits the differentiation of samples indicative of underlying high-grade cervical disease from samples that are indicative of benign proliferation, early-stage HPV infection, or mild dysplasia. By “early-stage HPV infection” is intended HPV infection that has not progressed to cervical dysplasia. As used herein, “mild dysplasia” refers to low-grade squamous intraepithelial lesion (LSIL) or cervical intraepithelial neoplasia 1 (CIN1) or both where no high-grade lesion is present. In this manner, the methods of the invention permit the accurate identification of high-grade cervical disease, even in cases mistakenly classified as normal, CIN1, LSIL, atypical squamous cells—undetermined significance (ASC-US), or atypical squamous cells—favor HSIL (ASC-H) by traditional Pap testing (i.e., “false negatives”). In some embodiments, the methods for diagnosing high-grade cervical disease are performed as a reflex test following an abnormal or atypical Pap smear. That is, the methods of the invention may be performed in response to a patient having an abnormal or atypical Pap smear result. In other aspects of the invention, the methods are performed as a primary screening test for high-grade cervical disease in the general population of women, just as the conventional Pap test is performed currently. In the latter instance, a slide would be simultaneously stained with the pap stain and with stains to detect biomarkers and the presence of absence of the biomarkers would determine the interpretation of the presence or absence of a high-grade lesion.

The cervical cell sample is stained with the Pap stain using well-known and commonly used methods. The sample is then exposed to a plurality of different fluorescent or transmission stain labeled biomarkers, at least one of the plurality of different labeled biomarkers operable for targeting at least one proliferative biomarker and at least one of the others of the plurality of different labeled biomarkers operable for labeling one or more of a plurality of different high-risk HPV strains when present. In some embodiments, several different strains of high-risk HPV are uniquely labeled such that each can be individually identified, and in other embodiments multiple high-risk HPV strains have the same label and are not distinguishable. Since many transmission stains also fluoresce, they can also be used as fluorescence labels as well, providing that their native fluorescence spectra can be distinguished from the other labels being used. Care must also be taken that the absorbance spectra of any transmission stains used do not coincide with the maxima of the fluorescence spectra of any of the other labels being used. In one embodiment of this invention, Fast Red stain is used to detect a cocktail of probes for identification of high-risk HPV strains and Fast Blue stain is used to detect the proliferative marker p16 and these stains, together with the PAP stain, are imaged in transmission.

Multispectral images of the sample are generated using signals obtained from the plurality of labeled biomarkers and the Pap stain. An acousto-optic tunable filter (AOTF) can be used for the multispectral imaging. AOTF has the advantage of high temporal resolution (e.g. wavelength switching speed) and spectral versatility. However, liquid crystal tunable filters, Sagnac-interferometer Fourier systems, tomographic imagers, “push-broom” imaging devices of all types, a collection of interference filters (including those placed as a thin-film mask placed over an imaging device (e.g. a CCD chip) such as in a Bayer pattern or in other types of masking patterns) or any other suitable spectral imaging technology may also generally be used.

The multi-spectral images can include images from both visible and near infrared wavelengths, determined by the specific choice of labels used. The specific choice of biomarker labels is governed by the need to be able to distinguish them from the underlying PAP stain being used. Through the use of spectral unmixing techniques well known in the art, it is possible to quantify the signals from the biomarkers even when their spectra overlap substantially with that of the PAP stain.

Automated analysis of the multispectral biomarker images determines from which locations higher magnification multispectral PAP images should be acquired and analyzed for subsequent review by a pathologist. The slide is scanned in its entirety, with multispectral images taken at each location in the scan. Using spectral pixel unmixing or other analysis techniques, the high-risk HPV and proliferative marker abundance images are obtained. The choice of appropriate reference spectra is essential for performing this analysis correctly and must take into account that a given label spectrum can change in the presence of the other labels. Post-processing of the abundance images using standard intensity thresholding and morphological criteria is often required to remove artifacts.

At locations positive for one or both of these biomarkers, a series of multispectral transmission images of the PAP-stained slide at higher magnification are acquired. The results at each such location presented to the pathologist for final review consist of an image of the PAP stained cells obtained by colorizing the PAP abundance image derived from these higher magnification images, with the ability to overlay images showing the regions of high risk HPV and proliferative marker positivity obtained from their respective abundance images derived from these higher magnification images. The pathologist has the ability to adjust the color balance of the PAP stain image to suit his or her taste. For regions in which there is no high-risk HPV or proliferative marker signal detectable, multispectral PAP transmission images are not acquired and data from these regions are not subject to pathologist review.

The use of a multiply-labeled single slide allows for improved sensitivity without compromising efficiency. In addition to its application to automated screening, the present disclosure facilitates the pathologist's understanding of the natural history of cervical HPV infections, and thus enables improved predictive skills for determining a women's risk of developing cervical cancer. Embodiments of the invention also help to relieve the ever increasing shortage of qualified cytopathologists required to perform conventional manual Pap screening.

As mentioned, the proposed invention is equally applicable to any disease state in which both a transmission stain and cell or tissue-based biomarkers are used to detect, quantify, stage or otherwise determine disease progression or treatment. A representative embodiment would be to the detection of breast cancer. For the detection of this cancer, pathologists conventionally examine the morphology of the excised tissue using H&E transmission stain. A number of biomarkers have been found very helpful in further establishing the presence, extent, and optimum treatment for the cancer, among them the overexpression of the proteins ER, PR and Her2-neu. Conventionally, each of these proteins is looked at using immunohistochemical staining on a separate slice of tissue. This requires using multiple slices of tissue that is sometimes in short supply, and requires the pathologist to view and then mentally superimpose images from multiple slides to come to the proper diagnosis and interpretation of the biomarker expression. The present invention would allow labeling of these probes through fluorescence or transmission staining together with the H&E staining on a single slide. In this embodiment, the multispectral imaging and subsequent analysis would be used to provide intensity images of each of the labeled biomarkers overlaid on a color H&E transmission image.

Thus, in various embodiments, the automated analysis of multispectral biomarker images includes steps of labeling a sample with a general stain and at least one specific label; acquiring one or more spectral images of the labeled sample; processing the acquired spectral images to permit the at least one specific label to be distinguished from the general stain; using the processed acquired images which distinguish the at least one specific label to identify at least one portion of the sample which requires further pathologist review or interpretation.

A sample can include a solution with single cells or groups of cells as well as tissues. The sample may be single cells and/or groups of cells that are spread onto a substrate such as a coverslip or microscope slide or may also include tissues, including sectioned tissues, that are mounted onto a substrate such as a coverslip or microscope slide.

A general stain includes stains such as hematoxylin (which stains acidic structures such as the DNA in the nucleus of cells) or eosin (which stains basic structures such as the cytoplasm), or combinations of such stains (such as Papanicolaou stain), which label all or most cells. A specific label is a label such as an antibody or nucleotide probe which is directed to a particular structure(s) or chemical(s), generally with a high degree of specificity. The specific labels in turn may be directly or indirectly coupled to a marker such as a fluorescent dye, or an enzyme or other means for generating a dye such as a localized precipitate (e.g. DAB). A specific label may have a marker (a dye or an enzyme or other means for generating a dye) directly coupled to the specific label or indirectly, for example a secondary antibody which binds to a specific label such as a primary antibody, where the secondary antibody has a marker attached thereto. Fluorescent probes that may be used include organic dyes such as DAPI, FITC, Rhodamine, Cyanine dyes, etc.; quantum dots, and phosphorescence probes. Transmission stains that may be used include Alcian blue, PAS (with and without diastase pretreatment), trichrome, reticulin, Prussian blue, gram stain, grocott methenamine silver, and DAB. In some embodiments, the transmission stains are imaged using fluorescence microscopy.

In a particular embodiment directed to screening cervical samples, the general stain may be Papanicolaou stain and specific labels may include high risk HPV DNA markers and antibodies directed the proliferative marker p16INK4a. Other potential proliferative markers include interleukin-2, Ki-67, MCM2, PCNA, and topoisomerase II alpha. Various strains of high risk HPV may be separately identified, including HP-16, HP-18, HP31, and HP-33.

The sample may be scanned manually or in an automated manner, stopping at random locations or at locations that are determined by the presence of one or more indicators, such as the detection of the presence of a cell, which in turn may be indicated by the detection of a general stain or a specific label. One or more locations on a sample may be examined and images collected therefrom, with the locations from which images are collected being recorded for later reference by a clinician or other individual, for additional imaging or for direct inspection of the sample.

One or more spectral images may be collected using a microscope system that is configured to collect images using fluorescence and/or transmitted light. The sample may be illuminated with UV, visible, and/or infrared light and images of the sample may be collected at varying emission wavelengths or bands of wavelengths. Illuminating and/or collecting images of the sample at varying wavelengths may be performed using bandpass filters, e.g. on a slider or filter wheel; using an acousto-optical tunable filter (AOTF) device; a liquid crystal tunable filter (LCTF); Sagnac-interferometer Fourier systems; tomographic imagers; “push-broom” imaging devices of all types; a collection of interference filters (including those placed as a thin-film mask placed over an imaging device (e.g. a CCD chip) such as in a Bayer pattern or in other types of masking patterns); or any other suitable spectral imaging technology may also generally be used.

So-called transmission dyes, including hematoxylin, eosin, DAB, TMB, and others (including various dyes included in Papanicolaou stain), can be imaged using either transmission or fluorescence microscopy, or combinations thereof. Using such procedures, a complete spectrum can be collected at each pixel of each image taken of the sample. When the sample contains multiple transmission or fluorescence labels, and in particular when those labels' spectra overlap, techniques such as spectral unmixing or other spectral analysis techniques can be used to separate out the individual spectra of each dye so that the spatial locations and intensities of each dye can be individually resolved in the sample. By separating out the individual spectra, the structures that are labeled by the general stain(s) and specific label(s) can be discerned. Thus, if the labeling of a particular structure indicates the possible presence of a disease state, the computer system can identify the individual cells, groups of cells, or regions of the tissue within the sample which potentially have the disease marker and which may need to be examined and interpreted by trained personnel.

In the particular embodiment of screening cervical samples, if a region of the sample is positive for one or both of high risk HPV DNA label or pl6INK4a then such a region or regions are brought to the attention of the trained personnel (e.g. technician, clinician, or pathologist) for further examination and analysis.

Methods for preparing samples, collecting images, and processing image data can be found in U.S. application Ser. No. 12/675,677, and U.S. Pat. No. 7,316,904, each of which is incorporated herein by reference in its entirety.

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments. Rather, the scope of the invention should be defined in accordance with the following claims and their equivalents.

Although the invention has been illustrated and described with respect to one or more implementations, equivalent alterations and modifications will occur to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

1. An automated screening method for detecting abnormalities in a sample, comprising:
staining a sample with a histologic or cytologic stain for transmission light microscopy to provide a stained sample;
exposing said stained sample to a plurality of differentially-labeled biomarkers, wherein each of said biomarkers is labeled with a distinct transmission stain or fluorescence probe;
at a first location, generating at least one multi-spectral image of said stained sample using signals obtained from said plurality of differentially-labeled biomarkers and said histologic or cytologic stain; and
automatically determining whether the first location requires further pathologist review or interpretation.

2. The method of claim 1, wherein said multi-spectral image includes wavelengths in both the visible range and near-IR range.

3. The method of claim 1, wherein generating at least one multi-spectral image comprises using an imaging system including at least one of an acousto-optic tunable filter (AOTF), a liquid crystal tunable filter (LCTF), a Sagnac-interferometer Fourier system, a tomographic imager, a “push-broom” imaging device, an imaging detector having a multispectral mask superimposed thereon, and a plurality of interference filters.

4. The method of claim 1, wherein said automatically determining comprises identifying a region within the first location in which abnormalities in the cell or tissue sample are suspected.

5. The method of claim 1, wherein the sample comprises a cell or a tissue.

6. The method of claim 5, wherein the sample comprises a cell and wherein the stained cells comprise a cytology preparation.

7. The method of claim 5, wherein the sample comprises a tissue and wherein the stained tissue comprises a histology preparation.

8. The method of claim 1, wherein the plurality of differentially-labeled biomarkers includes at least one of a nucleic acid and a protein.

9. The method of claim 8, wherein the nucleic acid sequence includes a nucleic acid of infectious origin.

10. The method of claim 1, wherein the sample comprises a cervical cell sample.

11. The method of claim 1, wherein the plurality of differentially-labeled biomarkers comprises a probe for proliferative marker p16 and a probe for high-risk HPV.

12. The method of claim 11, wherein the p16 probe is labeled with Fast Blue, the high-risk HPV probe is labeled with Fast Red, and the histologic or cytologic stain is Pap stain.

13. The method of claim 1, wherein said automatically determining comprises identifying a region within the first location which is positive for at least one of a proliferative marker and high-risk HPV.

14. The method of claim 1, wherein at least one of the plurality of differentially-labeled biomarkers is labeled with a transmission stain, and wherein generating at least one multi-spectral image of said stained sample is obtained from at least one of the histologic or cytologic stain and the differentially-labeled biomarker transmission stain using fluorescence microscopy.

15. An automatic Pap screening method for detecting abnormalities in a cervical cell sample, comprising:
staining a cervical cell sample with papanicolaou (Pap) stain to provide Pap stained cells;
exposing said Pap stained cells to a plurality of differentially-labeled biomarkers, at least one of said plurality of differentially-labeled biomarkers operable for labeling at least one proliferative marker and at least one other of said plurality of differentially-labeled biomarkers operable for labeling at least one of a plurality of different high-risk human papilloma virus (HPV) strains, wherein each of said biomarkers is labeled with a distinct transmission stain or fluorescence probe;
at a first location, generating at least one multi-spectral image of said Pap stained cells using signals obtained from said plurality of differentially-labeled biomarkers and said Pap stain, and
automatically determining whether the first location includes at least one of a high-risk HPV strain and a proliferative marker using said multi-spectral image.

16. The method of claim 15, wherein said multi-spectral image includes wavelengths in both the visible range and near-IR range.

17. The method of claim 15, wherein generating at least one multi-spectral image comprises using an imaging system including at least one of an acousto-optic tunable filter (AOTF), a liquid crystal tunable filter (LCTF), a Sagnac-interferometer Fourier system, a tomographic imager, a “push-broom” imaging device, an imaging detector having a multispectral mask superimposed thereon, and a plurality of interference filters.

18. The method of claim 15, provided said automatically determining determines that said high-risk HPV virus strains or proliferative markers are present in said sample,
obtaining transmission images of said Pap stained cells, and
identifying said transmission images as requiring review to determine whether cervical cancer is present.

19. The method of claim 15, wherein the sample comprises a cell or a tissue.

20. The method of claim 19, wherein the sample comprises a cell and wherein the stained cells comprise a cytology preparation.

21. The method of claim 19, wherein the sample comprises a tissue and wherein the stained tissue comprises a histology preparation.

22. The method of claim 15, wherein the plurality of differentially-labeled biomarkers includes at least one of a nucleic acid and a protein.

23. The method of claim 22, wherein the nucleic acid sequence includes a nucleic acid of infectious origin.

24. The method of claim 15, wherein the plurality of differentially-labeled biomarkers comprises a probe for proliferative marker p16.

25. The method of claim 24, wherein the p16 probe is labeled with Fast Blue, the high-risk HPV probe is labeled with Fast Red, and the first transmission stain is Pap stain.

26. The method of claim 15, wherein each of a plurality of high-risk HPV strains are uniquely labeled such that each can be individually identified.

27. The method of claim 15, wherein said automatically determining comprises identifying a region within the first location which is positive for at least one of the proliferative marker and the at least one of a plurality of different high-risk human papilloma virus (HPV) strains.

28. The method of claim 15, wherein at least one of the plurality of differentially-labeled biomarkers is labeled with a transmission stain, and wherein generating at least one multi-spectral image of said stained sample is obtained from at least one of the histologic or cytologic stain and the differentially-labeled biomarker transmission stain using fluorescence microscopy.
